(19) United States
(12) Patent Application Publication
(10) Pub. No.: US 2019/0117195 A1
(43) Pub. Date: Apr. 25, 2019

(54) Visualization of Ultrasound Vector Flow Imaging (VFI) Data

(71) Applicant: Analogic Canada Corporation, Saint-Laurent (CA)

(72) Inventors: Brandon Miles, Richmond (CA); Reza Zahiri, Vancouver (CA); Hani Eskandari, Vancouver (CA)

(73) Assignee: Analogic Canada Corporation, Saint-Laurent, QC (CA)

(21) Appl. No.: 16/086,515

(22) PCT Filed: Mar. 21, 2016
(86) PCT No.: PCT/IB2016/051579

§ 371 Date: Sep. 19, 2018

(51) Int. Cl. A61B 8/08; A61B 8/06; G06T 7/20; G06T 3/00

(52) U.S. Cl. CPC A61B 8/5207; A61B 8/06; A61B 8/0891; A61B 2562/0204; G06T 3/0093; A61B 2562/04; G06T 7/20

(57) ABSTRACT

A method of ultrasound imaging includes transmitting an ultrasound signal with an ultrasound transducer array. The method further includes receiving from the ultrasound transducer array electrical signals indicative of ultrasound echoes received by the ultrasound transducer array. The method further includes beamforming the electrical signals, which results in beamformed data. The method further includes processing the beamformed data, which generates an image. The image represents at least an anatomical vessel of interest. The method further includes processing the beamformed data, which generates flow direction data and flow magnitude data for blood cells flowing in a predetermined region of the anatomical vessel. The method further includes processing the flow direction data and the flow magnitude data, which creates a visualization of the flow direction data and the flow magnitude data for the entire predetermined region of the vessel. The method further includes visually presenting the image with the visualization superimposed thereover.

### TECHNICAL FIELD

The following generally relates to ultrasound imaging and more particularly to visualization of ultrasound vector flow imaging (VFI) data.

### BACKGROUND

Ultrasound imaging provides information about the interior of a subject. For example, ultrasound imaging can be used to generate an image of a blood vessel and estimate blood flow velocity of blood cells inside the blood vessel. One approach to estimating the blood flow velocity is vector flow imaging (VFI). VFI provides a direction and magnitude of the flow at every point in the image. This is in contrast to conventional color flow measurements that only provide a speed of flow along a single axis. Different VFI techniques have been introduced as a means to measure and display blood flow. VFI data contains detailed information on how the flow moves or changes over time. VFI can provide more complete information of the flow such as vortices, flow reversal and flow direction changes across the vessel.

Turbulent flow visualization is also used for diagnosing medical conditions. For example, patent ductus arteriosus, which occurs when the ductus arteriosus (a connection between the pulmonary artery and aorta) fails to close postnatally, is diagnosed by noticing a retrograde color flow jet in a Doppler flow image. Similarly, the quality of flow in an arteriovenous fistula (AVFs) is monitored frequently to measure the direction of flow and to diagnose any turbulent flow in the region. Guidelines have recommended that such monitoring be performed on all mature arteriovenous fistulas (AVFs) on a weekly basis and prior to starting dialysis for each patient. Making turbulent flow easier to visualize has the potential to make such monitoring and diagnosis easier and more effective.

Real-time visualization for VFI includes 1-D color coding based on magnitude of flow combined with arrows on a grid that show local direction of the flow, and 2-D color coding methods that show both magnitude and direction of flow with colors while using arrows as an additional information to show the direction and the magnitude. These methods introduce limitations when flow is complex and dynamic as they are inherently local. They are local in that they only display flow information at a single point without providing a global view of the flow. They are static in that they only show static flow information at each time point, as opposed to information about the dynamics of flow over time. It is also difficult to visualize turbulent flow with existing methods. Moreover, 2-D color maps are not intuitive and are difficult to interpret for the end user, and they are not well-suited for concurrently visualizing magnitude and direction of flow.

Methods that employ contours and fixed arrows have similar limitations when it comes to complex and dynamic flow since they are local and static. B-Flow imaging (BFI) has also been used for visualization of flow. In BFI, ultrasound speckle patterns are directly visualized to show the movement of the blood. BFI does not offer vector flow measurement quantitatively, just visualization of motion. Vector Flow Mapping uses stationary arrows to show the flow vector which is another local visualization method. Vector Projectile Imaging combines high frame rate imaging with moving arrows showing flow. This method is not suitable for real-time visualization since the data needs to be acquired using ultrafast imaging and processed offline. Pathlet visualization requires ultrafast imaging with 2-D speckle tracking to acquire vector data. However, it is not suitable for real-time visualization since data needs to be acquired using ultrafast imaging.

### SUMMARY

Aspects of the application address the above matters, and others.

In one aspect, a method of ultrasound imaging includes transmitting an ultrasound signal with an ultrasound transducer array. The method further includes receiving from the ultrasound transducer array electrical signals indicative of ultrasound echoes received by the ultrasound transducer array. The method further includes beamforming the electrical signals, which results in beamformed data. The method further includes processing the beamformed data, which generates an image. The image represents at least an anatomical vessel of interest. The method further includes processing the beamformed data, which generates flow direction data and flow magnitude data for blood cells flowing in a predetermined region of the anatomical vessel. The method further includes processing the flow direction data and the flow magnitude data, which creates a visualization of the flow direction data and the flow magnitude data for the entire predetermined region of the vessel. The method further includes visually presenting the image with the visualization superimposed thereover.

In another aspect, a computer readable storage medium is encoded with computer readable instructions, which, when executed by a processer, cause the processor to: receive, from an ultrasound transducer array, electrical signals indicative of ultrasound echoes received by the ultrasound transducer array, generate beamformed data by beamforming the electrical signals, generate an image by processing the beamformed data, generate vector flow imaging data by processing the beamformed data, wherein the vector flow imaging data includes a flow direction data and a flow magnitude data for blood cells flowing in a predetermined region of an anatomical vessel, create a visualization of the flow direction data and the flow magnitude data for the entire predetermined region of the vessel with the vector flow imaging data, and display the image with the visualization overlaid thereover.

In another aspect, an ultrasound imaging console includes receive circuitry (106) configured to receive from an ultrasound transducer array electrical signals indicative of ultrasound echoes received by the ultrasound transducer array. The console further includes a beamformer configured to beamform the electrical signals. The console further includes an image processor configured to generate an image by processing the beamformed electrical signals. The console further includes a vector flow imaging processor configured to determine a flow direction and a flow magnitude by processing the beamformed electrical signals using a vector flow imaging algorithm. The console further includes a visualization processor configured to generate a visualization of the determined flow direction and of the flow magnitude. The console further includes a display configured to display the generated image with the generated visualization of the determined flow direction and of the flow magnitude superimposed over the displayed image.

Those skilled in the art will recognize still other aspects of the present application upon reading and understanding the attached description.

### BRIEF DESCRIPTION OF THE DRAWINGS

The application is illustrated by way of example and not limited by the figures of the accompanying drawings, in which like references indicate similar elements and in which:

FIG. 1 schematically illustrates an example ultrasound imaging console with at least a VFI processor and a visualization processor;

FIG. 2 illustrates an example of a B-mode image;

FIG. 3 illustrates an example MPMS visualization with gray scale representing flow magnitude and arrows representing global flow;

FIG. 4 illustrates an example of the B-mode image of FIG. 2 with the MPMS visualization of FIG. 3 superimposed thereover;

FIG. 5 illustrates an example of a B-mode image;

FIG. 6A illustrates an example of flow direction arrows representing local flow at single points;

FIG. 6B illustrates an example flow magnitude color map;

FIG. 7 illustrates a prior art example with the B-mode image of FIG. 5 with the flow direction arrows of FIG. 6A and the flow magnitude color map of FIG. 6B superimposed thereover;

FIG. 8 illustrates an example of a B-mode image;

FIG. 9 illustrates an example DIS visualization with gray scale representing flow magnitude and streamlines, created via line integral convolution algorithm with white noise, representing global flow;

FIG. 10 illustrates an example of the B-mode image of FIG. 8 with the DIS visualization of FIG. 9 superimposed thereover;

FIG. 11 illustrates an example of a B-mode image;

FIG. 12 illustrates an example DIS visualization with gray scale representing flow magnitude and streamlines, created via line integral convolution algorithm with sparse noise, representing global flow;

FIG. 13 illustrates an example of the B-mode image of FIG. 11 with the DIS visualization of FIG. 12 superimposed thereover;

FIG. 14 illustrates an example of a B-mode image;

FIG. 15 illustrates an example DIS visualization with gray scale representing flow magnitude and streaklines, created via line integral convolution algorithm with white noise, representing global flow;

FIG. 16 illustrates an example of the B-mode image of FIG. 14 with the DIS visualization of FIG. 15 superimposed thereover;

FIG. 17 illustrates an example of a B-mode image;

FIG. 18 illustrates an example DIS visualization with gray scale representing flow magnitude and streaklines, created via line integral convolution algorithm with sparse noise, representing global flow;

FIG. 19 illustrates an example of the B-mode image of FIG. 17 with the DIS visualization of FIG. 18 superimposed thereover;

FIG. 20 illustrates an example of a B-mode image;

FIG. 21 illustrates an example FTV visualization with gray scale representing flow magnitude and lines representing global flow;

FIG. 22 illustrates an example of the B-mode image of FIG. 17 with the FTV visualization of FIG. 18 superimposed thereover; and

FIG. 23 illustrates a method for visualization flow magnitude and global flow determined based on VFI data with a B-mode image.

### DETAILED DESCRIPTION

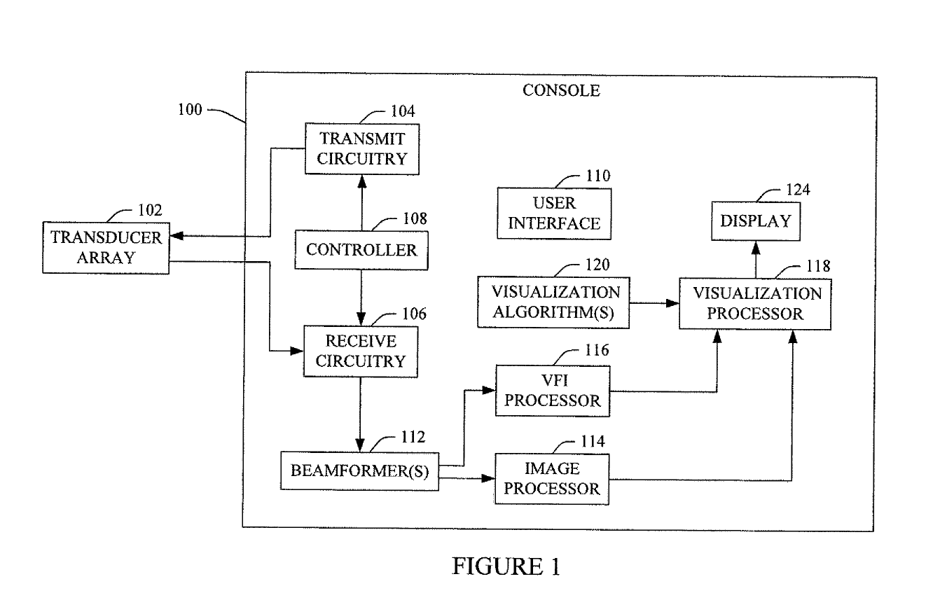

Initially referring to FIG. 1, an example system including ultrasound imaging console 100 in connection with a transducer array 102 is schematically illustrated.

The transducer array 102 includes a one-dimensional (1-D) array or a two dimensional (2-D) array of transducer elements, which are configured to transmit ultrasound signals, receive echo signals and generate electrical signals indicative of the received echo signals. Examples of 1-D arrays include 16, 32, 64, 128, 256, etc., and examples of 2-D arrays include 32×32, 64×64, etc., and/or other dimension arrays, including circular, elliptical, rectangular, irregular, etc. The transducer array 102 can be linear, curved, and/or otherwise shaped, fully populated, sparse, etc.

Transmit circuitry 104 generates a set of pulses that are conveyed to the transducer array 102. The set of pulses invokes a set of the transducer elements to transmit ultrasound signals. In the illustrated embodiment, transmit circuitry 104 generates a set of pulses which produce a transmit signal suitable at least for vector flow imaging (VFI). Receive circuitry 106 receives electrical signals from the transducer array 102, which are indicative of echoes received by elements of the transducer array 102. The echoes, generally, are a result of the interaction between the transmitted ultrasound signals and structure such as flowing blood cells in a vessel, organ cells, soft tissue, etc.

A controller 108 controls one or more of the transmit circuitry 104 or receive circuitry 106. Such control can be based on available modes of operation (e.g., velocity flow imaging, B-mode imaging, velocity flow imaging +B-mode imaging, etc.) of the console 100. In addition, such control can be based on one or more signals indicative of input from a user, a default configuration, etc. A user interface (UI) 110 produces the one or more signals indicative of the input from a user. The UI 110 may include one or more input devices (e.g., a button, a knob, a slider, a touch pad, etc.) and/or one or more output devices (e.g., a display screen, lights, a speaker, etc.).

A beamformer(s) 112 processes the electrical signals and produces data used to generate at least an image and a vector velocity estimate. For a B-mode image, the beamformer 112 processes the signals by applying time delays, weighting the channels, and summing the weighted delayed signal, and/or otherwise beamforming the signals. For transverse oscillation (TO) VFI, the beamformer 112 beamforms beams to produce spatial lateral in-phase (I) and quadrature (Q) components. For this, a transverse oscillation is introduced in the ultrasound field, and this oscillation generates received signals that depend on the transverse oscillation. The basic idea is to create a double-oscillating pulse-echo field and use a particular apodization profile(s) in receive. Suitable apodization functions are discussed in Jensen et al., “A New Method for Estimation of Velocity Vectors,” IEEE Trans. Ultrason., Ferroelec., Freq. Contr., vol. 45, pp. 837-851, 1998, and Udesen et al., “Investigation of Transverse Oscillation Method,” IEEE Trans. Ultrason., Ferroelec., Freq. Contr., vol. 53, pp. 959-971, 2006. The beamformer(s) 112 can process the electrical signals to produce data for other vector flow approaches.

Returning to FIG. 1, an image processor 114 processes the beamformed data and generates a sequence of focused, coherent echo samples along focused scanlines of a scanplane, or a B-mode image. The image processor 114 may also be configured to process the scanlines to lower speckle and/or improve specular reflector delineation via spatial compounding and/or perform other processing such as FIR filtering, IIR filtering, etc. The B-mode image can be displayed, e.g., in a graphical user interface (GUI), which allows a user to selectively rotate, scale, manipulate, take measurements on, etc. the displayed data. This can be through a mouse, a keyboard, a touch-screen and/or the like.

A vector flow imaging (VFI) processor 116 also processes the beamformed data. In one instance, this includes processing the beamformed data using a TO approach and determining from the processed data one or more velocity components such as an axial/depth velocity component, a lateral velocity component, and/or an elevation velocity component. Examples of suitable velocity processing are described in patent U.S. Pat. No. 6,148,224 A, titled “Apparatus and method for determining movements and velocities of moving objects,” and filed Dec. 30, 1998, patent U.S. Pat. No. 6,859,659 A, titled “Estimation of vector velocity,” and filed May 10, 2000, the entirety of which is incorporated herein by reference, and patent application US 2014/0257103 A1, titled “Three Dimensional (3D) Transverse Oscillation Vector Velocity Ultrasound Imaging,” and filed Oct. 11, 2011, the entirety of which is incorporated herein by reference.

A visualization processor 118 receives both the images and the vector velocity estimates, processes this data via a visualization algorithm(s) 120 to create a visualization of flow magnitude and direction, and visually presents the visualization via a display 122. As described in greater detail below, the visualization processor 118, in one instance, superimposes or overlays graphical indicia indicative of static and/or dynamic flow over a B-mode image. This visualization can provide a global view of the flow throughout the entire image and/or a local view of the flow at particular points in the image. In one example, graphical indicia for flow measurements can show global flow characteristics such as vortices, flow reversal, flow direction changes across an entire predetermined portion of a vessel of interest. In one instance, this provides an intuitive and informative visualization for clinicians. Furthermore, this approach can be used for real-time (i.e., during scanning as ultrasound signals are transmitted and echoes are received and processed) visualizations and/or off-line data ultrasound visualizations, including 2-D, 3-D, 4-D and/or other flow visualization.

It is to be appreciated that one or more of the velocity processor 116 and the visualization processor 120 can be implemented by a processor (e.g., a central processing unit, a microprocessor, etc.) executing a computer readable instruction embedded or stored on non-transitory computer readable medium (which excludes transitory computer readable medium) such as physical memory and/or carried by transitory computer readable medium such as a carrier wave, signal, etc. Additionally or alternatively, the velocity processor 116 and the visualization processor 120 can be implemented by a processor are implemented by the processor executing instructions carried by transitory computer readable medium (which excludes non-transitory computer readable medium) such as a carrier wave, a signal, etc.

Furthermore, the console 100 can be part of a portable system on a stand with wheels, a system residing on a tabletop, and/or other system in which the transducer array 102 is housed and physically supported in a probe or the like and the console 100 is part of an apparatus separate therefrom. In this instance, the transducer array 102 and the console 100 transfer information there between via a wired and/or wireless connection/communication channel via complementary communication interfaces. In another instance, the transducer array 102 and the console 100 are housed and physically supported in a same apparatus such as within a single enclosure hand-held ultrasound scanning device.

Examples of suitable visualization algorithm(s) 120 include, but are not limited to, a massless particle motion simulation (MPMS) algorithm, a direct image synthesis (DIS) algorithm, a flow trace visualization (FTV) algorithm, a combination thereof, and/or other visualization algorithm. Examples of these algorithms are described next.

With the MPMS algorithm, the visualization processor 120 injects virtual particles into the flow pattern. The particles propagate based on the underlying flow vector data for better visualization of the motion. The method allows the user to virtually see the blood as it flows through the vessel. The seeding of the particles for injecting new particles can be random and/or based on a uniform distribution. Random seeding may provide a more realistic visualization of the flow. Different particles can be used for this purpose. Particles can be simple geometric shapes such as squares, circles, points, etc., other geometric shapes such as arrows, lines, cones, pictographs, virtual blood cells, glyphs, etc., and/or other graphical indicia. These particles can also have a life span, dying out after a set number of frames, seconds or steps.

With the MPMS algorithm, the visualization processor 120 can control (e.g., set and/or change) the flow visualization speed, including scaling (e.g., slowing down and/or speeding up) the flow. In one instance, this adjusts the speed so that the user can visually perceive the flow, e.g., for flow that would otherwise be too fast for the user to visually perceive. In one example, this scaling factor can be a ratio between display rate (˜30-60 Hz) in real-time and the actual acquisition rate (in the kHz range). This allows the user to visualize the flow in slow motion where the particles may be moving too fast to be recognizable in the display. This can be done in real-time without slowing down the speed of imaging (e.g., decreasing the frame rate). This represents an improvement in the technology.

With the MPMS algorithm, the visualization processor 120 can additionally or alternatively perform one or more of the following variations and/or other variations. The visualization processor 120 can adjust color, transparency, orientation, etc. of particles based on flow magnitude, flow direction, flow variance, flow voracity, flow turbulence, etc. An example of this is colorized ellipses oriented in the flow direction, with the lengths of the major and minor axes adjusted based on the flow variance. The visualization processor 120 can combine/fuse this approach with other visualization methods such as conventional 1-D method, static arrows, etc. The visualization processor 120 can enhance the data via lighting effects, such as specular and diffuse reflections. Again, the visualization approach can be employed with 2-D, 3-D, 4-D and/or other flow visualization.

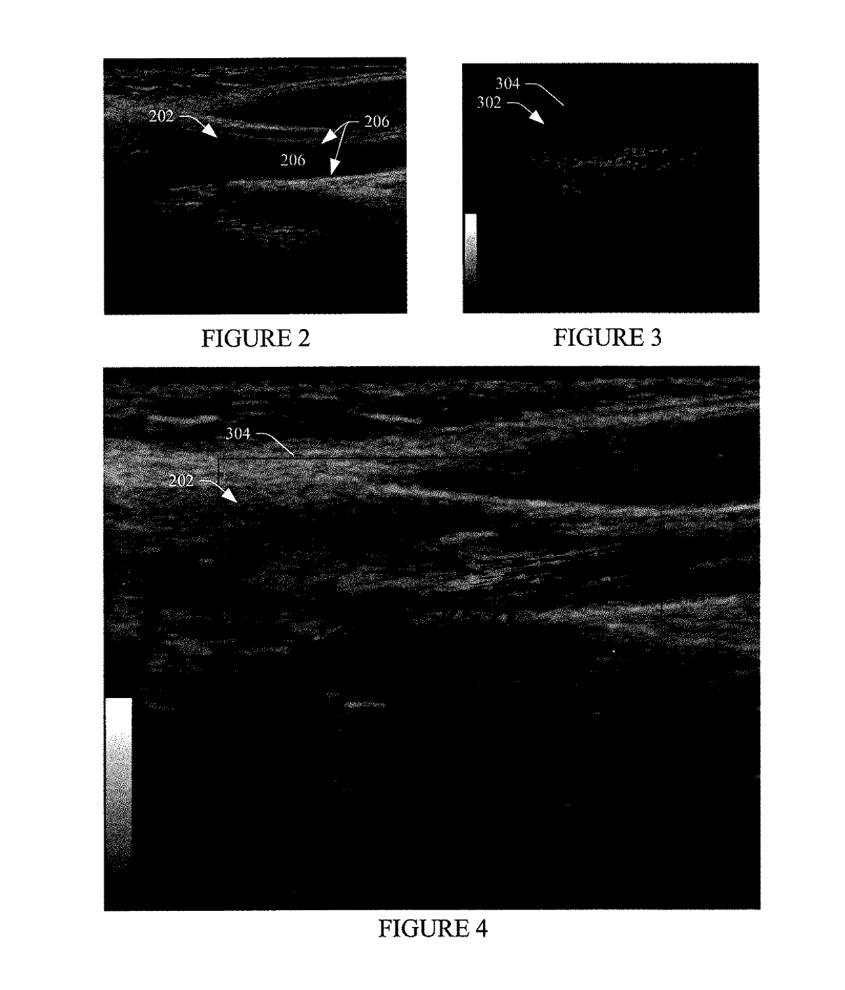

FIGS. 2-4 visualization examples using the MPMS algorithm.

FIG. 2 show a B-mode image with a vessel 202, including a vessel wall 204 and a lumen 206. FIG. 3 shows an MPMS visualization 302 for flow within in the lumen 206 for an entire predetermined region 304, using gray scale to represent flow magnitude and arrows to represent global flow. FIG. 4 shows the B-mode image with the MPMS visualization 302 superimposed over the vessel 202.

With the DIS algorithm, the visualization processor 120 warps or distorts a certain image in the direction of the flow. This has the effect of blowing or smearing the input image in the direction of the flow. It can be done statically (considering only the current frame) for an instantaneous view of the flow or dynamically (considering the flow over multiple previous frames) for time varying flow showing the direction of flow over time. An example DIS algorithm is the line integral convolution (LIC) algorithm. This algorithm utilizes a noise texture as the input image and distorts it based on the flow. Both white noise and sparse noise can be used as the input image. The method can also be extended by using other types of input images.

With the DIS algorithm, the visualization processor 118 can additionally or alternatively perform one or more of the following variations and/or other variations. The visualization processor 120 can control the flow visualization speed, as described with the MPMS algorithm. The visualization processor 120 can apply color coding, transparency, etc. to images based on flow magnitude, flow direction, flow variance, flow vorticity, flow turbulence, etc. The visualization processor 120 can combine/fuse this approach with other visualization methods such as conventional 1-D method, static arrows, etc. The visualization processor 120 can enhance the data via lighting effects, such as specular and diffuse reflections. Again, the visualization approach can be employed with 2-D, 3-D, 4-D and/or other flow visualization.

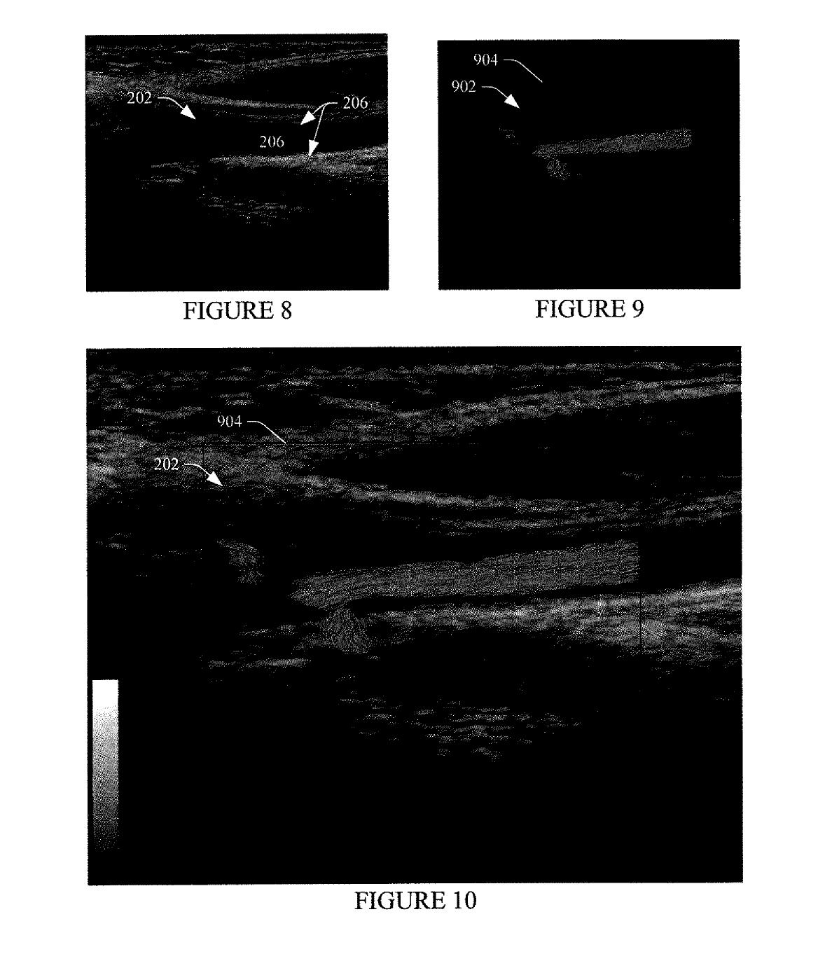

FIGS. 8-10, 11-13, 14-16 and 17-19 show visualization examples using the DIS algorithm.

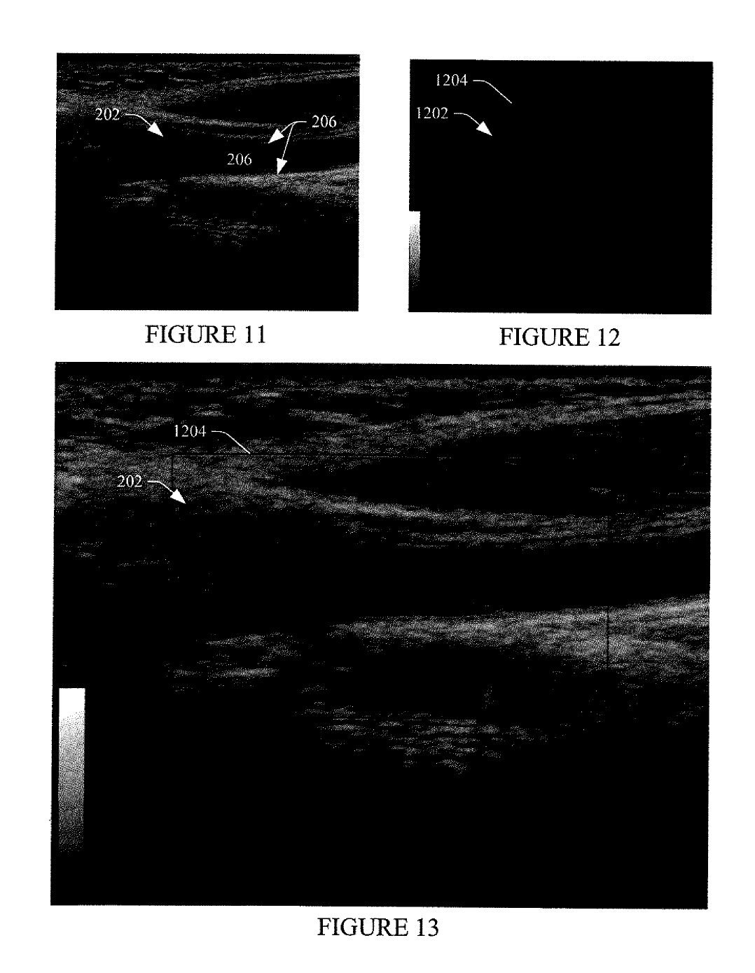

FIG. 8 show the B-mode image of FIG. 2. FIG. 9 shows a DIS visualization 902 for flow within in the lumen 206 for an entire predetermined region 904, using gray scale to represent flow magnitude and streamlines, created via LIC with white noise, to represent global flow. FIG. 10 shows the B-mode image with the DIS visualization 902 superimposed over the vessel 202. FIG. 11 show the B-mode image of FIG. 2. FIG. 12 shows a DIS visualization 1202 for flow within in the lumen 206 for an entire predetermined region 1204, using gray scale to represent flow magnitude and streamlines, created via LIC with sparse noise, to represent global flow. FIG. 10 shows the B-mode image with the DIS visualization 1202 superimposed over the vessel 202.

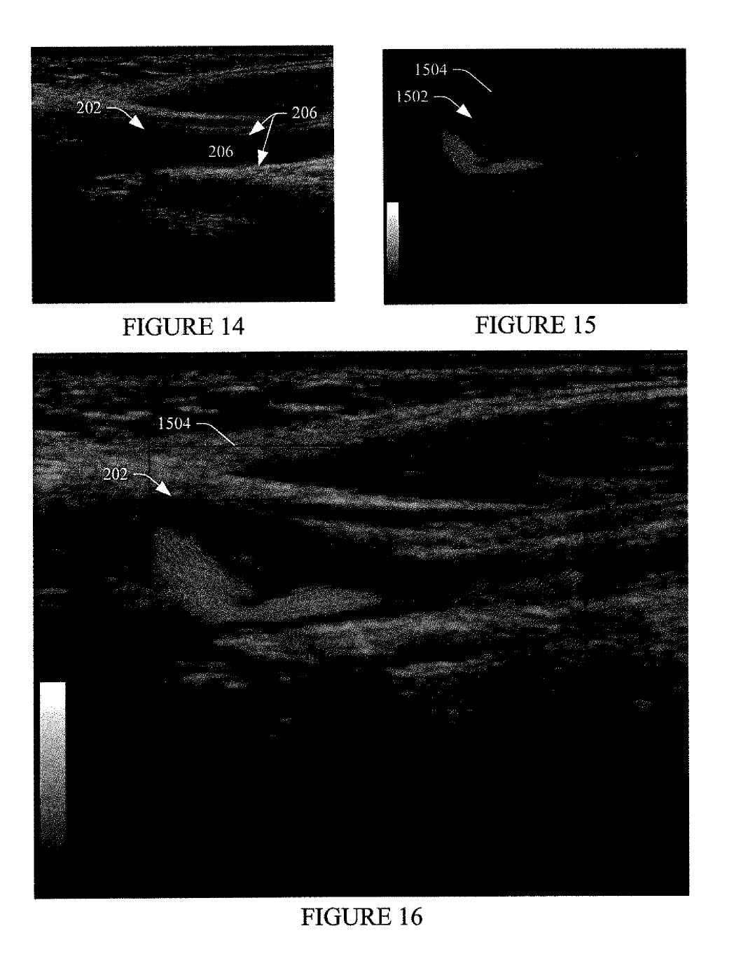

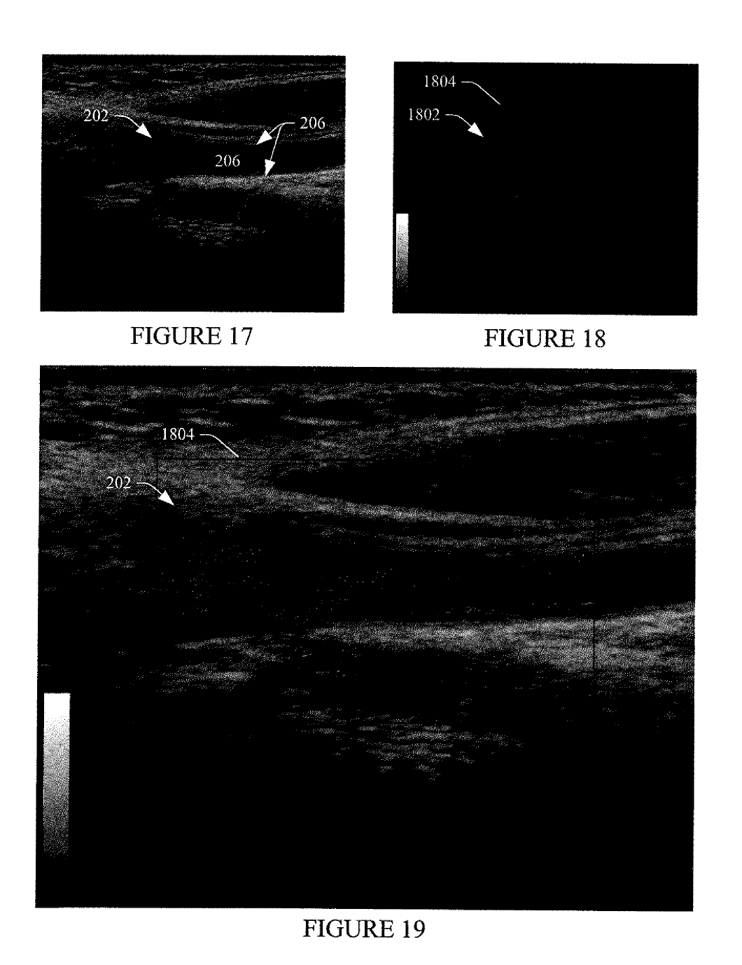

FIG. 14 shows the B-mode image of FIG. 2. FIG. 15 shows a DIS visualization 1502 for flow within in the lumen 206 for an entire predetermined region 1504, using gray scale to represent flow magnitude and streaklines, created via time varying LIC with white noise, to represent global flow. FIG. 16 shows the B-mode image with the DIS visualization 1502 superimposed over the vessel 202. FIG. 17 show the B-mode image of FIG. 2. FIG. 18 shows a DIS visualization 1802 for flow within in the lumen 206 for an entire predetermined region 1804, using gray scale to represent flow magnitude and streaklines, created via time varying LIC with sparse noise, to represent global flow. FIG. 19 shows the B-mode image with the DIS visualization 1802 superimposed over the vessel 202.

With the FTV algorithm, the visualization processor 120 seeds lines in the flow direction to generate their trace. These lines follow the pattern of the flow. These lines can have a life span, die out and be reseeded after a set number of frames, seconds or steps. This allows the flow to be visualized by tracing its path. Seeding of the traces can be done on a fixed grid or a random grid. With the FTV algorithm, the visualization processor 120 can additionally or alternatively perform one or more of the below variations and/or other variations.

With the FTV algorithm, the visualization processor 120 can apply color coding, transparency, etc. to images and widths of lines based on flow magnitude, flow direction, flow variance, etc. The visualization processor 120 can combine this approach with other visualization methods such as flow trace+conventional 1D method+arrows, etc. The visualization processor 120 can enhance the data via lighting effects, such as specular and diffuse reflections. Again, the visualization approach can be employed with 2-D, 3-D, 4-D and/or other flow visualization. This can be with either lines in 3-D or 2-D dimensional ribbons rendered in 3-D. When using ribbons, ribbons can also be twisted based on flow turbulence or voracity.

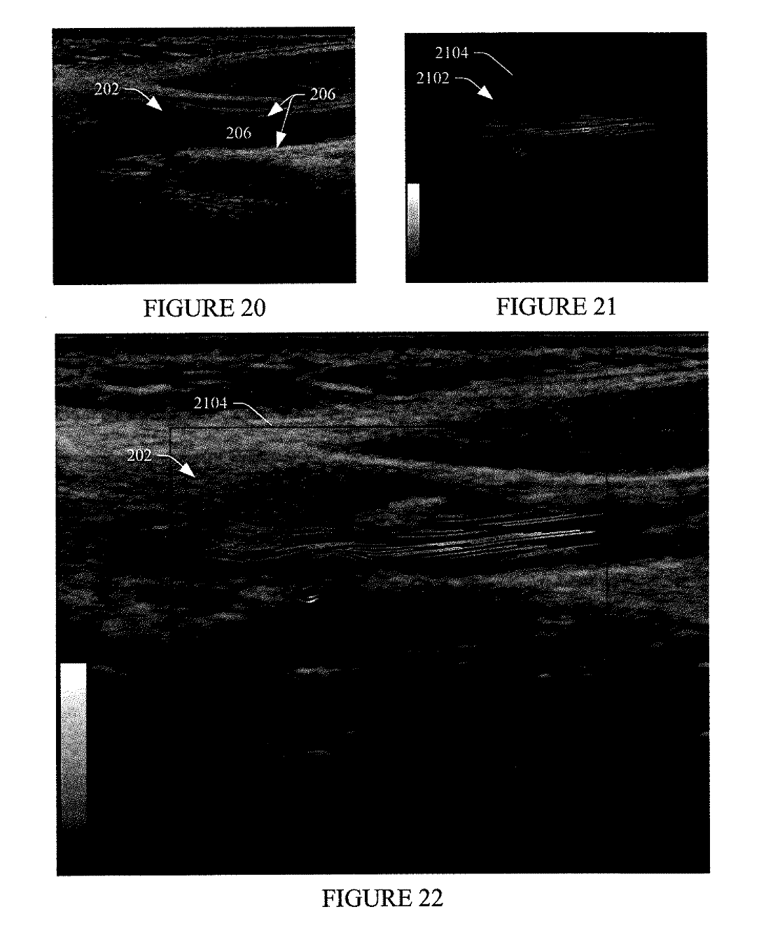

FIGS. 20-22 show visualization examples using the FTV algorithm. FIG. 20 show the B-mode image of FIG. 2. FIG. 21 shows a FTV visualization 2102 for flow within in the lumen 206, using gray scale to represent flow magnitude and lines to represent flow direction. FIG. 22 shows the B-mode image with the FTV visualization 2102 superimposed over the vessel 202.

In one instance, the flow is updated via an estimate of the new position. This can be done in a linear manner or using higher order estimate i.e. RungeKutta, etc. In one instance, an effect of higher order integration is a more accurate flow path. This estimation would be used in all the visualization methods if it was implemented i.e. MPMS, DIS and/or FTV.

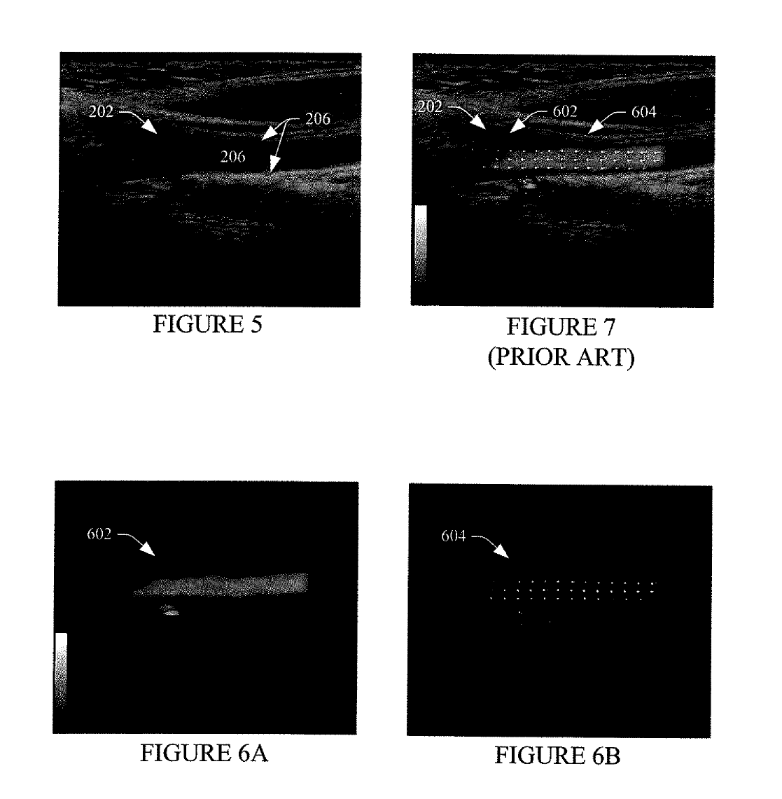

For comparison, FIGS. 5, 6A, 6B and 7 show a prior art visualization combining a magnitude color map and arrows for local point based flow. FIG. 5 shows the B-mode image of FIG. 2. FIG. 6A shows a flow magnitude color map 602 in the lumen 206 for certain points. FIG. 6B shows flow direction arrows 604 for the lumen 206. FIG. 7 shows the B-mode image of FIG. 5 with the magnitude color map 602 and the local flow direction arrows 604 superimposed over the vessel 202.

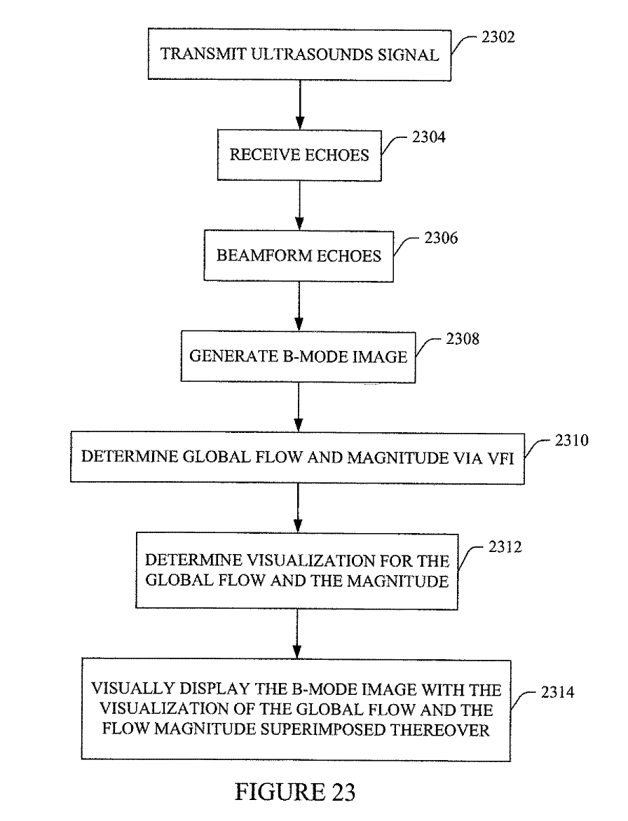

FIG. 23 illustrates a method for visualization flow magnitude and global flow direction determined based on VFI data with a B-mode image.

It is to be understood that the following acts are provided for explanatory purposes and are not limiting. As such, one or more of the acts may be omitted, one or more acts may be added, one or more acts may occur in a different order (including simultaneously with another act), etc.

At 2302, an ultrasound signal is transmitted into a field of view.

At 2304, echoes, in response to the ultrasound signal, are received by a transducer array.

At 2306, the echoes are beamformed.

At 2308, a B-mode image is generated with the beamformed echoes.

At 2310, flow direction and magnitude are determined via VFI with the beamformed echoes, as described herein and/or otherwise.

At 2312, visualizations of the global flow direction and flow magnitude are determined, as described herein and/or otherwise.

At 2314, the B-mode image is visually displayed with a visualization of the global flow and flow magnitude superimposed thereover.
The methods described herein may be implemented via one or more processors executing one or more computer readable instructions encoded or embodied on computer readable storage medium such as physical memory which causes the one or more processors to carry out the various acts and/or other functions and/or acts. Additionally or alternatively, the one or more processors can execute instructions carried by transitory medium such as a signal or carrier wave.

The application has been described with reference to various embodiments. Modifications and alterations will occur to others upon reading the application. It is intended that the invention be construed as including all such modifications and alterations, including insofar as they come within the scope of the appended claims and the equivalents thereof.

1. A method of ultrasound imaging, comprising:
transmitting an ultrasound signal with an ultrasound transducer array;
receiving from the ultrasound transducer array electrical signals indicative of ultrasound echoes received by the ultrasound transducer array;
beamforming the electrical signals, which generates beamformed data;
processing the beamformed data, which generates an image, wherein the image represents at least an anatomical vessel of interest;
processing the beamformed data, which generates flow direction data and flow magnitude data for blood cells flowing in a predetermined region of the anatomical vessel;
processing the flow direction data and the flow magnitude data, which creates a visualization of the flow magnitude data and the flow direction data for the entire predetermined region of the vessel; and
visually presenting the image with the visualization superimposed thereover.

2. The method of claim 1, wherein the processing of the flow direction data and the flow magnitude data includes using a massless particle motion simulation algorithm in which graphical indicia is injected into the predetermined region of the vessel and propagates within the predetermined region of the vessel based on the flow direction data.

3. The method of claim 2, further comprising
seeding the graphical indicia with one of a random distribution or a uniform distribution.

4. The method of claim 2, wherein graphical indicia are removed from the display after at least one of a predetermined time from injection or a predetermined number of frames from injection.

5. The method of claim 2, further comprising:
controlling a flow speed of the graphical indicia independent of a frame rate as the electrical signals are generated and received.

6. The method of claim 1, wherein the processing of the flow direction data and the flow magnitude data includes using a direct image synthesis algorithm, which warps the predetermined region of the vessel in a direction of the flow.

7. The method of claim 6, further comprising:
warping only a current frame, providing an instantaneous view of the flow.

8. The method of claim 6, further comprising:
warping multiple frames, providing a time varying flow showing the direction of flow over time.

9. The method of claim 6, wherein the flow direction data and the flow magnitude data is processed using a line integral convolution algorithm.

10. The method of claim 9, wherein the line integral convolution algorithm warps one of a white noise input image or a sparse noise input image.

11. The method of claim 1, wherein the processing of the flow direction data and the flow magnitude data includes using a flow trace visualization algorithm, which seeds lines in a flow direction to generate their trace.

12. The method of claim 11, further comprising:
controlling a flow speed of the lines independent of a frame rate as the electrical signals are generated and received.

13. The method of claim 11, further comprising
seeding the lines based on a random or fixed grid.

14. The method of claim 11, wherein a line is removed from the display after at least one of a predetermined time of creation or a predetermined number of frames from creation.

15. A computer readable medium embedded with computer executable instructions, which, when executed by a processor of a computer, causes the processor to:
receive, from an ultrasound transducer array, electrical signals indicative of ultrasound echoes received by the ultrasound transducer array;
generate beamformed data by beamforming the electrical signals;
generate an image by processing the beamformed data;
generate vector flow imaging data by processing the beamformed data, wherein the vector flow imaging data includes a flow direction data and a flow magnitude data for blood cells flowing in a predetermined region of an anatomical vessel;
create a visualization of the flow direction data and the flow magnitude data for the entire predetermined region of the vessel with the vector flow imaging data; and
display the image with the visualization overlaid thereover.

16. The computer readable medium of claim 15, wherein the processer creates the visualization based on one or more of a massless particle motion simulation algorithm, a direct image synthesis algorithm, and a flow trace visualization algorithm.

17. The computer readable medium of claim 15, wherein the visualization includes at least one of color coding and transparency based on flow magnitude, direction, variance, vorticity, and turbulence.

18. The computer readable medium of claim 15, wherein the visualization includes a lighting effect, including at least one of specular reflection and diffuse reflection.

19. The computer readable medium of claim 15, wherein the visualization is one of a 2-D, a 3-D, or a 4-D flow visualization.

20. The computer readable medium of claim 15, wherein the visualization shows a flow variance.

21. The computer readable medium of claim 15, wherein the visualization shows a flow vorticity.

22. The computer readable medium of claim 15, wherein the visualization shows a flow turbulence.

23. The computer readable medium of claim 20, wherein the visualization includes ribbons that are twisted based on the flow turbulence or the flow vorticity.

24. An ultrasound imaging console, comprising:
receive circuitry configured to receive, from an ultrasound transducer array, electrical signals indicative of ultrasound echoes received by the ultrasound transducer array;
a beamformer configured to beamform the electrical signals;
an image processor configured to generate an image by processing the beamformed electrical signals;
a vector flow imaging processor configured to determine a flow direction and a flow magnitude by processing the beamformed electrical signals using a vector flow imaging algorithm;
a visualization processor configured to generate a visualization of the determined flow direction and of the flow magnitude; and
a display configured to display the generated image with the generated visualization of the determined flow direction and of the flow magnitude superimposed over the displayed image.

25. The console of claim 24, wherein the vector flow imaging processor uses a transverse oscillation approach to determine the flow direction and magnitude.

26. The console of claim 24, wherein the visualization algorithm is a massless particle motion simulation algorithm.

27. The console of claim 24, wherein the visualization algorithm is a flow trace visualization algorithm.

28. The console of claim 24, wherein the visualization algorithm is a direct image synthesis algorithm.
